(19) United States
(12) Patent Application Publication
(10) Pub. No.: US 2021/0322247 A1
(43) Pub. Date: Oct. 21, 2021

(54) TREATMENT PROTOCOL FOR LOW BACK SYNDROME

(71) Applicant: Guy P. Curtis, San Diego, CA (US)

(72) Inventor: Guy P. Curtis, San Diego, CA (US)

(21) Appl. No.: 16/850,517

(22) Filed: Apr. 16, 2020

(51) Int. Cl. A61H 1/02; A61P 19/02; A61K 31/18; A61K 31/133; A61K 31/325

(52) U.S. Cl. CPC A61H 1/0222; A61P 19/02; A61K 31/18; A61H 2203/0493; A61K 31/325; A61H 2203/0456; A61K 31/133

(57) ABSTRACT

A protocol for the treatment of lumbosacral spine disease requires the combination of physical and pharmaceutical therapies. For the physical therapy aspect of the present invention, an inversion table is used to periodically impose traction forces of the spine of a patient. Preferably, this is done with the patient lying supine on the inversion table in a head down orientation, with the inversion table inclined at a predetermined angle in a range that includes 45°, for a predetermined time interval that includes 3 to 4 minutes. A pharmaceutical therapy is also clinically prescribed, in conjunction with the physical therapy aspect, that requires ingesting a nonsteroidal anti-inflammatory drug (NSAID), a muscle relaxant, and an antiarrhythmic drug in accordance with a predetermined regimen.

### FIELD OF THE INVENTION

The present invention pertains generally to protocols that combine physical and pharmaceutical therapies for the treatment of a disease. More particularly, the present invention pertains to protocols for the treatment of lumbosacral spine disease. The present invention is particularly, but not exclusively, useful as a treatment for lumbosacral spine disease that periodically imposes traction forces on the spine of a patient, in conjunction with a daily regimen of selected pharmaceuticals.

### BACKGROUND OF THE INVENTION

It is well known that lumbosacral spine disease causes low back, hip and/or leg pain. In general, it is thought by many that these ailments typically result from different causes that are directly or indirectly associated with physical activity. More specifically, these causes can include: i) the movement of discs or vertebral bodies that compress inflamed nerves exiting from the spinal cord; ii) the pull from muscles on vertebral bodies that affect spinal alignment; and iii) the repetitive firing of inflamed motor nerves that cause muscles attached to the spine to contract and thereby affect spinal alignment and the compression of sensory nerves.

Heretofore, noninvasive evaluations of lumbosacral spine disease have been accomplished using radiographic technologies that involve x-ray, gamma rays or similar radiation. Although surgeries that are performed to correct radiographically detected alignment problems have been generally successful, adverse symptoms will often persist for extended periods of time after surgery. Quite likely, these symptoms persist because radiographs taken from a supine patient do not accurately reflect the structure of the spine when the patient is otherwise sitting, or erect, or engaged in some form of activity. For these reasons, the present invention envisions that a satisfactory relief for a patient, from the symptoms of lumbosacral disease, will often require pain medications, acupuncture or some other form of continuing therapy for their discomfort.

In light of the above, it is an object of the present invention to provide a protocol for the treatment of lumbosacral disease that includes a combination of physical and pharmaceutical therapies. Another object of the present invention is to provide a treatment for lumbosacral spine disease that periodically imposes traction forces on the spine of a patient, in conjunction with a daily regimen of selected pharmaceuticals. Still another object of the present invention is to provide a symptom driven protocol for relieving the pain associated with lumbosacral spine disease which can be monitored to evaluate efficacy in terms of symptom alleviation. It is also an object of the present invention to provide a protocol for the treatment of lumbosacral disease that is easy to follow, is efficacious, and is cost effective.

### SUMMARY OF THE INVENTION

In accordance with the present invention, a protocol for treating a lumbosacral spine disease of a patient requires the combination of physical and pharmaceutical therapies. For the physical therapy aspect of the protocol, a device is preferably used once or twice daily to apply a traction force along the spine of the patient that will open up the space between vertebrae. Depending on the needs of a patient, the traction force will have a predetermined magnitude, and it will be applied for a predetermined time interval. For the pharmaceutical therapy aspect of the protocol, the patient is expected to ingest a nonsteroidal anti-inflammatory drug (NSAID), a muscle relaxant, and an antiarrhythmic drug in accordance with a clinically prescribed regimen.

As envisioned for the present invention, a device for applying a traction force on the patient will preferably be an inversion table of a type well known in the pertinent art. When an inversion table is used, it is the weight W of the patient under the influence of gravity that will cause the traction force to be generated. Using an inversion table, the physical therapy aspect of the protocol begins with the patient being supported and held horizontally on the inversion table in a supine position. He/she is then rotated on the inversion table from the horizontal orientation around a horizontal axis, through a predetermined rotation angle θ. With the inversion table at the rotation angle θ the patient is held in a head down orientation for a predetermined time interval. At the end of this time interval the inversion table is rotated back to a horizontal orientation and the physical therapy aspect of the protocol is ended.

An alternate embodiment of the present invention uses a traction bed of a type well known in the pertinent art for purposes of the protocol. For the alternate embodiment, rather than relying on the patient's body weight W, predetermined weights can be arranged on the traction bed and connected to the patient (e.g. legs) for use as an alternative device for purposes of generating a traction force.

As envisioned for purposes of the present invention, the rotation angle θ for an inversion table will preferably be in a range between 40° and 60°. Further, the predetermined time interval will preferably be in a range between 2 minutes and 5 minutes (e.g. 3-4 minutes). It is also to be noted that for a patient having a weight W, the predetermined angle θ is preferably selected to create a traction force on the patient that is greater than approximately 0.5 W. Using these general parameters, the physical therapy aspect of the protocol is accomplished at least once or twice daily.

It is an important aspect of the present invention that for a complete protocol, which may last several months, there should be a continuing, systematic clinical monitoring of the patient. Specifically, the severity of the symptoms associated with lumbosacral spine disease that are experienced by the patient needs to be periodically evaluated (e.g. monthly). The purpose here is to ascertain the degree to which these symptoms have been alleviated. With this monitoring, changes in the severity of symptoms can be used as an indicator of protocol efficacy.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a side elevation view of a patient held on an inversion table at a predetermined angle θ for the purpose of generating a traction force on the spine of the patient; and

FIG. 2 is a presentation of an exemplary model of a selective process for prescribing a patient-specific pharmaceutical regimen that can be incorporated into the protocol of the present invention.

### DESCRIPTION OF THE PREFERRED EMBODIMENTS

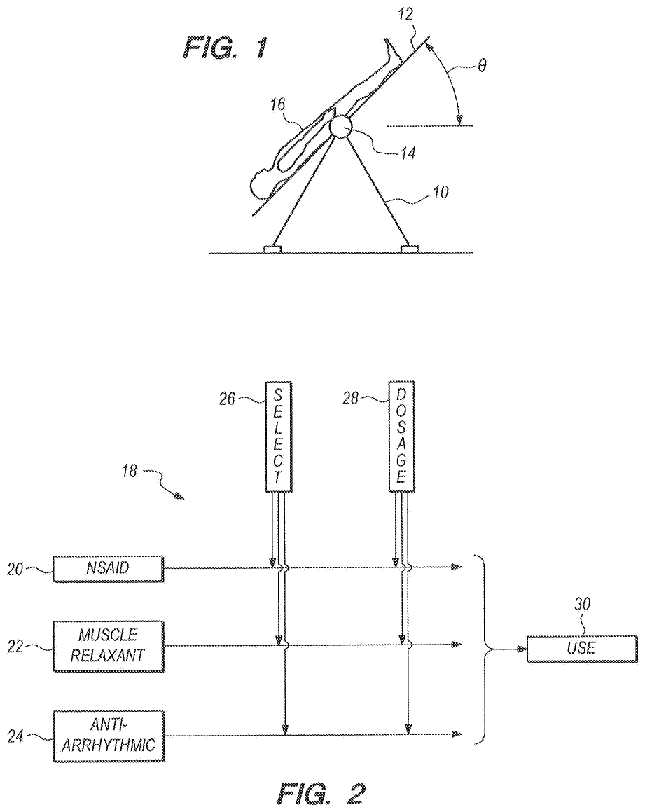

Referring initially to FIG. 1 is will be seen that a protocol in accordance with the present invention requires the use of a traction device, such as the inversion table which is shown in FIG. 1 and designated 10. As shown, it will be appreciated that the inversion table 10 includes a platform 12 which can be rotated about a horizontal axis 14 that is established by the inversion table 10. In an operation of the inversion table 10, a patient 16 is preferably positioned on the platform 12 in a supine position. The patient 16 can then be rotated with the platform 12 through a rotation angle θ around the horizontal axis 14 to impose a traction force on the spine of the patient 16. Preferably, the rotation angle θ is in a range between 40° and 60°.

Once the patient 16 is positioned in a head down orientation at the angle θ, as shown in FIG. 1, he/she will be held in this orientation for a predetermined time interval. Typically, the rotation angle θ will be in a range between 40° and 60°, and the time interval will be in a range between 2-5 minutes. For a patient of weight W, an orientation within these parameters will typically establish a traction force on the spine of the patient that will be greater than about 0.5 W during the physical therapy portion of the protocol.

With specific regard to the pharmaceutical therapy aspect of the protocol of the present invention, FIG. 2 presents a process for prescribing a patient-specific pharmaceutical regimen that is generally designated 18. In detail, the regimen 18 requires consideration of three types of pharmaceuticals, with each type having a particular therapeutic activity. These pharmaceuticals include: 1) a nonsteroidal anti-inflammatory drug (NSAID) 20 for decreasing neuronal inflammation; 2) a muscle relaxant 22 for blunting muscle spasms associated with neuronal inflammation; and 3) an antiarrhythmic drug 24 for decreasing the repetitive firing of motor neurons in order to decrease traction on vertebral bodies and improve alignment of the spine during physical activity.

For purposes of the present invention, several different pharmaceuticals within each type of pharmaceutical may be appropriately used. Moreover, it is appreciated by the present invention that each pharmaceutical will individually have a specific dosage efficacy. Accordingly, as indicated in FIG. 2, the preparation of the pharmaceutical regimen 18 includes a selection step 26 that requires prescribing certain pharmaceuticals, as needed. For example, a nonsteroidal anti-inflammatory drug (NSAID) 20 is selected from the group including celecoxib. Similarly, a muscle relaxant 22 is selected from the group including methocarbamol, and an antiarrhythmic drug 24 is selected from the group including sotalol or mexiletine. In each instance, the selection of a pharmaceutical is made on an as needed basis.

FIG. 2 further indicates that a prescription of the pharmaceutical regimen 18 also includes a dosage step 28 for each selected pharmaceutical. The consequence here is that after the selection step 26 and the dosage step 28 a pharmaceutical regimen is completed and is ready for a use 30 by the patient 16.

While the particular Treatment Protocol for Low Back Syndrome as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A protocol for the treatment of lumbosacral spine disease of a patient which comprises the steps of:
providing an inversion table having a platform for supporting and holding the patient thereon in a supine position, and wherein the inversion table defines a horizontal axis for measuring a rotation of the platform around the horizontal axis through a rotation angle θ measured from a horizontal orientation of the platform;
rotating the platform of the inversion table through a predetermined rotation angle θ to an orientation where the patient is held head down for a predetermined time interval; and
selecting a pharmaceutical for ingestion by the patient in accordance with a clinically prescribed regimen, wherein the pharmaceutical is selected from the group including a nonsteroidal anti-inflammatory drug, a muscle relaxant, and an antiarrhythmic drug.

2. The protocol of claim 1 wherein the rotation angle θ is in a range between 40° and 60°.

3. The protocol of claim 1 wherein the patient has a weight W and the predetermined angle θ creates a traction force on the patient greater than 0.5 W.

4. The protocol of claim 1 wherein the predetermined time interval is in a range between 2 minutes and 5 minutes.

5. The protocol of claim 1 wherein the nonsteroidal anti-inflammatory drug is selected from the group consisting of celecoxib.

6. The protocol of claim 1 wherein the muscle relaxant is selected from the group consisting of methocarbamol.

7. The protocol of claim 1 wherein the antiarrhythmic drug is selected from the group consisting of sotalol or mexiletine.

8. The protocol of claim 1 wherein the rotating step is accomplished at least once daily.

9. The protocol of claim 1 further comprising the step of periodically reviewing the severity of symptoms experienced by the patient to ascertain a degree of alleviation of the symptoms as an indicator of protocol efficacy.

10. The protocol of claim 9 wherein the step of reviewing the severity of symptoms is conducted monthly.

11. A protocol for treating a lumbosacral spine disease of a patient which comprises the steps of:
applying a traction force along the spine of the patient, wherein the traction force has a predetermined magnitude and is applied for a predetermined time interval; and
selecting a pharmaceutical for ingestion by the patient in accordance with a clinically prescribed regimen, wherein the pharmaceutical is selected from the group including a nonsteroidal anti-inflammatory drug, a muscle relaxant, and an antiarrhythmic drug.

12. The protocol of claim 11 further comprising the steps of:
providing an inversion table having a platform for supporting and holding a patient thereon in a supine position, and wherein the inversion table defines a horizontal axis for measuring a rotation of the platform around the horizontal axis through a rotation angle θ measured from a horizontal orientation of the platform; and
rotating the platform of the inversion table through a predetermined rotation angle θ to an orientation where the patient is held head down for a predetermined time interval.

13. The protocol of claim 12 wherein the rotation angle θ is in a range between 40° and 60°.

14. The protocol of claim 12 wherein the patient has a weight W and the predetermined angle θ creates a traction force on the patient greater than 0.5 W.

15. The protocol of claim 12 wherein the rotating step is accomplished at least once daily.

16. The protocol of claim 11 wherein the predetermined time interval is in a range between 2 minutes and 5 minutes.

17. The protocol of claim 11 further comprising the step of periodically reviewing the severity of symptoms experienced by the patient to ascertain a degree of alleviation of the symptoms as an indicator of protocol efficacy.

18. The protocol of claim 17 wherein the step of reviewing the severity of symptoms is conducted monthly.

19. The protocol of claim 11 further comprising the step of providing a traction bed for supporting the patient in a supine position during the applying step.

20. The protocol of claim 19 wherein the patient has a weight W and the traction force has a predetermined magnitude greater than 0.5 W and the traction force is applied for a predetermined time interval in a range between 2 minutes and 5 minutes.
